(19) United States
(12) Patent Application Publication
(10) Pub. No.: US 2007/0227547 A1
(43) Pub. Date: Oct. 4, 2007

(54) Treatment of the vertebral column

(75) Inventor: Hai H. Trieu, Cordova, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/354,314

(22) Filed: Feb. 14, 2006

(51) Int. Cl. A61B 19/00

(52) U.S. Cl. 128/898

(57) ABSTRACT

A method is provided for treating a spinal condition. The method includes introducing a biological treatment into an area of a vertebral column, and mechanically unloading the treated area by applying a load-bearing device to the anterior region, the anterior column region, the posterior region, or the spinous process region of the vertebral column.

### BACKGROUND

The present application relates generally to treatment of the vertebral column, for example, repairing or regenerating an area of the vertebral column, or reducing or preventing degeneration of an area of the vertebral column.

Disease, degradation, and trauma of the spine can lead to various conditions that require treatment to maintain, stabilize, or reconstruct the vertebral column. For example, degeneration of the facet joints and/or the intervertebral discs due to aging and/or trauma can lead to pain, neurological deficit and/or loss of motions that require treatment to maintain, stabilize, reconstruct and/or regenerate the degenerated levels. Repair/regeneration of such levels via a biological approach is technically challenging at least in part because of the high loading environment present in such levels. Reducing or preventing degeneration of an area of the vertebral column can be similarly challenging.

### SUMMARY

The present application relates generally to treatment of the vertebral column, for example, repairing or regenerating an area of the vertebral column, or reducing or preventing degeneration of an area of the vertebral column.

In one embodiment, a method of treating a vertebral column includes introducing a biological treatment into an area of a vertebral column, and at least partially mechanically unloading the treated area. In one aspect, the treated area is mechanically unloaded by applying a load-bearing device to at least one region of the vertebral column. In certain aspects, the load-bearing device is applied to an anterior region, an anterior column region, a posterior region, or a spinous process region of the vertebral column. In one aspect, a method of treating a vertebral column includes introducing a biological treatment into a facet joint in a vertebral column, and at least partially mechanically unloading the treated facet joint. In one aspect, the treated facet joint is at least partially mechanically unloaded by applying a load-bearing device to a posterior region of the vertebral column adjacent to the treated area.

In another embodiment, a method for treating a motion segment of a vertebral column includes accessing a portion of the patient's spinal column, implanting a load-bearing device into the motion segment, and injecting a biological treatment into the motion segment. The load-bearing device at least partially mechanically unloads the motion segment. In one such embodiment, the motion segment of the vertebral column is intact.

Additional embodiments are provided in the following description and the attached drawings.

### BRIEF DESCRIPTION OF THE DRAWINGS

FIG. 1 is a sagittal view of a motion segment of a vertebral column.

FIG. 2 is a superior view of a vertebral body depicted in FIG. 1.

FIGS. 3-4 illustrate methods for applying a biological treatment to a facet joint in a vertebral column.

FIGS. 5-6 illustrate methods for applying a biological treatment to a disc space in a vertebral column.

FIGS. 7A-7F illustrate methods for applying a biological treatment to a vertebral body and/or an endplate.

FIGS. 8A-8C illustrate alternative methods for applying a biological treatment to a disc space in a vertebral column.

FIG. 9 is a sagittal view of a motion segment of a vertebral column to which a biological treatment has been applied in combination with a mechanical unloading device.

### DETAILED DESCRIPTION

The present disclosure relates generally to treatment of the vertebral column, for example, repairing or regenerating an area of the vertebral column, or reducing or preventing degeneration of an area of the vertebral column.

Certain embodiments describe methods for treating motion segments of the spinal column and components thereof. Such embodiments include but are not limited to treating facet joints, intervertebral discs, vertebral bodies and endplates using a biological approach in combination with a mechanical unloading device that is at least partially load-bearing with respect to the treated area such that it at least partially unloads the treated area.

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

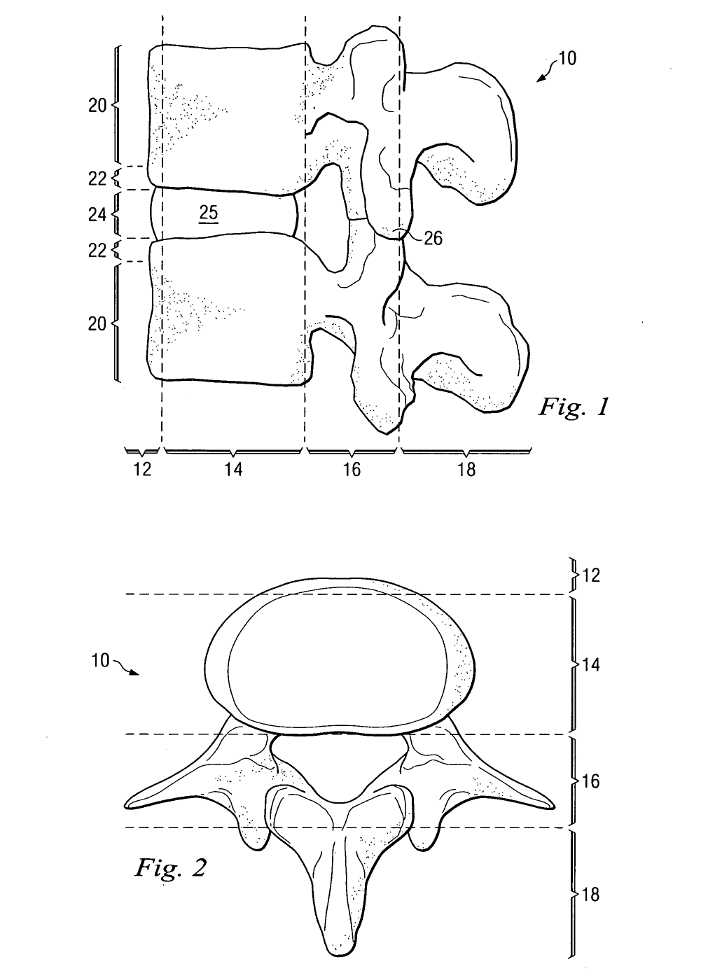

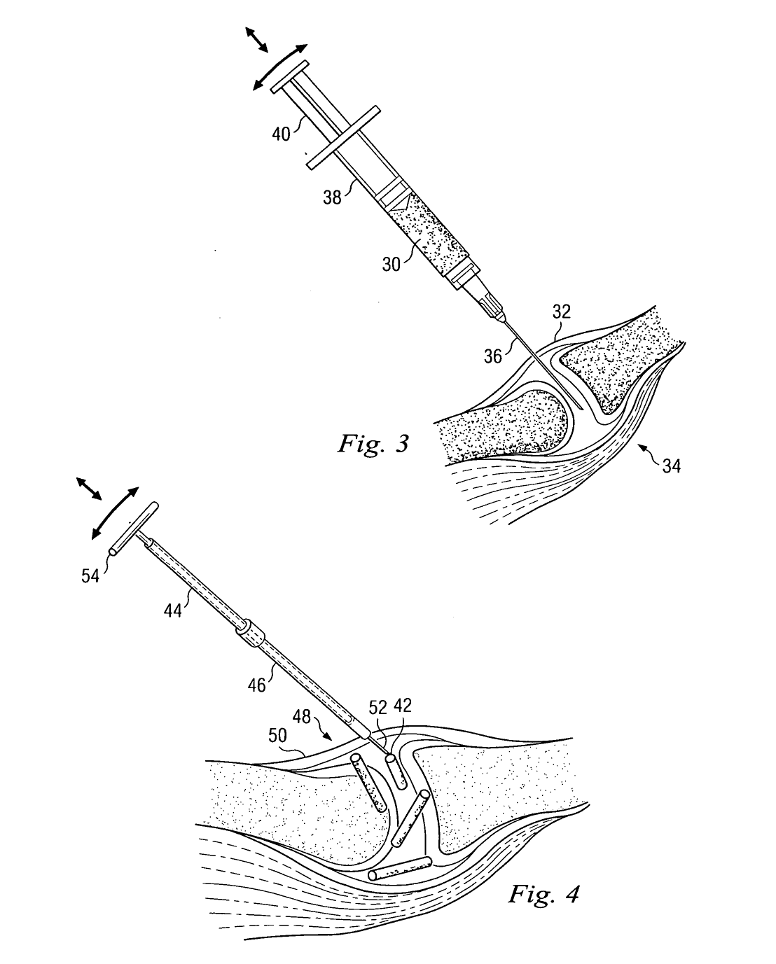

Referring now to FIGS. 1 and 2, the reference numeral 10 refers to a motion segment of a vertebral column. Motion segment 10 comprises an intervertebral disc 25 and a facet joint 26. Motion segment 10 may be considered as having several regions extending from anterior to posterior. These regions include an anterior region 12, an anterior column region 14, a posterior region 16, and a spinous process region 18. The anterior column region 14 may be further considered to have several regions extending longitudinally along the column. These regions include a vertebral body region 20, an endplate region 22, and a disc space region 24. Disc space region 24 includes the nucleus and annulus forming intervertebral disc 25.

Any of the regions illustrated in FIGS. 1 and 2 may benefit from a biological treatment as described herein. In certain embodiments, the biological treatment is non-load bearing. In certain aspects, a non-load bearing biological treatment comprises a composition that is applied without an associated support or structure. Treatment/treating of the vertebral column includes repair and/or regeneration of a degenerated area of the vertebral column, and/or reduction or prevention of degeneration of an area of the vertebral column. Methods for treating the vertebral column with a biological treatment and a device that is at least partially load-bearing with respect to the treated area such that the device at least partially mechanically unloads the treated area are described herein.

As used herein, a “biological treatment” includes but is not limited to a “biologically active component”, with or without a “biological additive”.

A “biologically active component” includes but is not limited to anti-cytokines; cytokines; anti-interleukin-1 components (anti-IL-1); anti-TNF alpha; “growth factors”; LIM mineralization proteins; “stem cell material”; autogenic chondrocytes; allogenic chondrocytes, such as those described in U.S. Patent Application Publication No. 2005/0196387, the entire disclosure of which is incorporated herein by reference; autogenic chondrocytes with retroviral viral vector or plasmid viral vector; allogenic chondrocytes with retroviral viral vector or plasmid viral vector; and fibroblasts. The acronym “LIM” is derived from the three genes in which the LIM domain was first described. The LIM domain is a cysteine-rich motif defined by 50-60 amino acids with the consensus sequence CX₂CX₁₆₋₂₃HX₂CX₂CX₂CX₁₆₋₂₁CX₂(C/H/D), which contains two closely associated zinc-binding modules. LIM mineralization proteins include but are not limited to those described in U.S. Patent Application Publication No. 2003/0180266 A1, the disclosure of which is incorporated herein by reference. “Growth factors” include but are not limited to transforming growth factor (TGF)-beta 1, TGF-beta 2, TGF-beta 3, bone morphogenetic protein (BMP)-2, BMP-3, BMP-4, BMP-6, BMP-7, BMP-9, fibroblast growth factor (FGF), platelet derived growth factor (PDGF), insulin-like growth factor (ILGF); human endothelial cell growth factor (ECGF); epidermal growth factor (EGF); nerve growth factor (NGF); and vascular endothelial growth factor (VEGF). “Anti-IL-1” components include but are not limited to those described in U.S. Patent Application Publication Nos. 2003/0220283 and 2005/0260159, the entire disclosures of which are incorporated herein by reference. “Stem cell material” includes but is not limited to dedifferentiated stem cells, undifferentiated stem cells, and mesenchymal stem cells. “Stem cell material” also includes but is not limited to stem cells extracted from marrow, which may include lipo-derived stem cell material and adipose-derived stem cell material, such as described in U.S. Publication Nos. 2004/0193274 and 2005/0118228, each of which is incorporated herein by reference. “Stem cell material” also includes but is not limited to stem cells derived from adipose tissue as described in U.S. Patent Application Publication Nos. 2003/0161816, 2004/0097867 and 2004/0106196, each of which is incorporated herein by reference.

A “biologically active component” also includes but is not limited to cartilage derived morphogenetic protein (CDMP); cartilage inducing factor (CIP); proteoglycans; hormones; and matrix metalloproteinases (MMP) inhibitors, which act to inhibit the activity of MMPs, to prevent the MMPs from degrading the extracellular matrix (ECM) produced by cells within the nucleus pulposus of the disc. Exemplary MMP inhibitors include but are not limited to tissue inhibitors, such as TIMP-1 and TIMP-2. Certain MMP inhibitors are also described in U.S. Patent Application Publication No. 2004/0228853, the entire disclosure of which is incorporated herein by reference.

A “biologically active component” also includes but is not limited to allogenic or xenogenic disc annulus material, such as described in U.S. Patent Application Publication No. 2005/0043801, the entire disclosure of which is incorporated herein by reference; biologic tissues, such as those described in U.S. Patent Application Publication No. 2003/0004574, the entire disclosure of which is incorporated herein by reference; an activated tissue graft, such as described in U.S. Patent Application Publication No. 2005/0136042, the entire disclosure of which is incorporated herein by reference; an engineered cell comprising a nucleic acid for encoding a protein or variant thereof, such as a BMP, a LIM mineralization protein, or an SMAD protein as described in U.S. Patent Application Publication Nos. 2003/0219423 and 2003/0228292, the entire disclosures of which are incorporated herein by reference; and a recombinant human bone morphogenetic protein, such as described in U.S. Patent Application Publication No. 2004/0024081, the entire disclosure of which is incorporated herein by reference.

As used herein, a “biological additive” includes but is not limited to “biomaterial carriers”, “therapeutic agents”, “liquids” and “lubricants.”

“Biomaterial carriers” include but are not limited to collagen, gelatin, hyaluronic acid, fibrin, albumin, keratin, silk, elastin, glycosaminoglycans (GAGs), polyethylene glycol (PEG), polyethylene oxide (PEO), polyvinyl alcohol (PVA) hydrogel, polyvinyl pyrrolidone (PVP), co-polymers of PVA and PVP, other polysaccharides, platelet gel, peptides, carboxymethyl cellulose, and other modified starches and celluloses. Collagen includes but is not limited to collagen-based material, which may be autogenic, allogenic, xenogenic or of human-recombinant origin, such as the collagen-based material described in U.S. Patent Application Publication Nos. 2004/0054414 and 2004/0228901, the entire disclosures of which are incorporated herein by reference.

“Therapeutic agents” include but are not limited to nutrients, analgesics, antibiotics, anti-inflammatories, steroids, antiviricides, vitamins, amino acids and peptides. Nutrients include but are not limited to substances that promote disc cell survival, such as glucose and pH buffers, wherein the pH buffer provides a basic environment in the disc space, which preferably will be a pH of about 7.4. Analgesics include but are not limited to hydrophilic opoids, such as codeine, prodrugs, morphine, hydromorphone, propoxyphene, hydrocodone, oxycodone, meperidine and methadone, and lipophilic opoids, such as fentanyl. Antibiotics include but are not limited to erythromycin, bacitracin, neomycin, penicillin, polymyxin B, tetracyclines, viomycin, chloromycetin and streptomycins, cefazolin, ampicillin, azactam, tobramycin, clindamycin and gentamycin.

“Liquids” include but are not limited to water, saline and radio-contrast media. Radio-contrast media includes but is not limited to barium sulfate, or a radio contrast dye, such as sodium diatrizoate (HYPAQUE™).

“Lubricants” include but are not limited to hyaluronic acid, a salt of hyaluronic acid, sodium hyaluronate, glucosaminoglycan, dermatan sulfate, heparin sulfate, chondroitin sulfate, keratin sulfate, synovial fluid, a component of synovial fluid, vitronectin and rooster comb hyaluronate.

A biological treatment may be introduced to an area of a vertebral column, such as a motion segment, by any method and in any form appropriate for such introduction. For example, the biological treatment can be injected, deposited, or applied, as a solution, a suspension, emulsion, paste, a particulate material, a fibrous material, a plug, a solid, porous, woven or non-woven material, or in a dehydrated or rehydrated state. Suitable forms for a biological treatment and suitable methods for injecting a biological treatment include those described in U.S. Patent Application Publication Nos. 2005/0267577, 2005/0031666, 2004/0054414, and 2004/0228901, each of which is incorporated herein by reference.

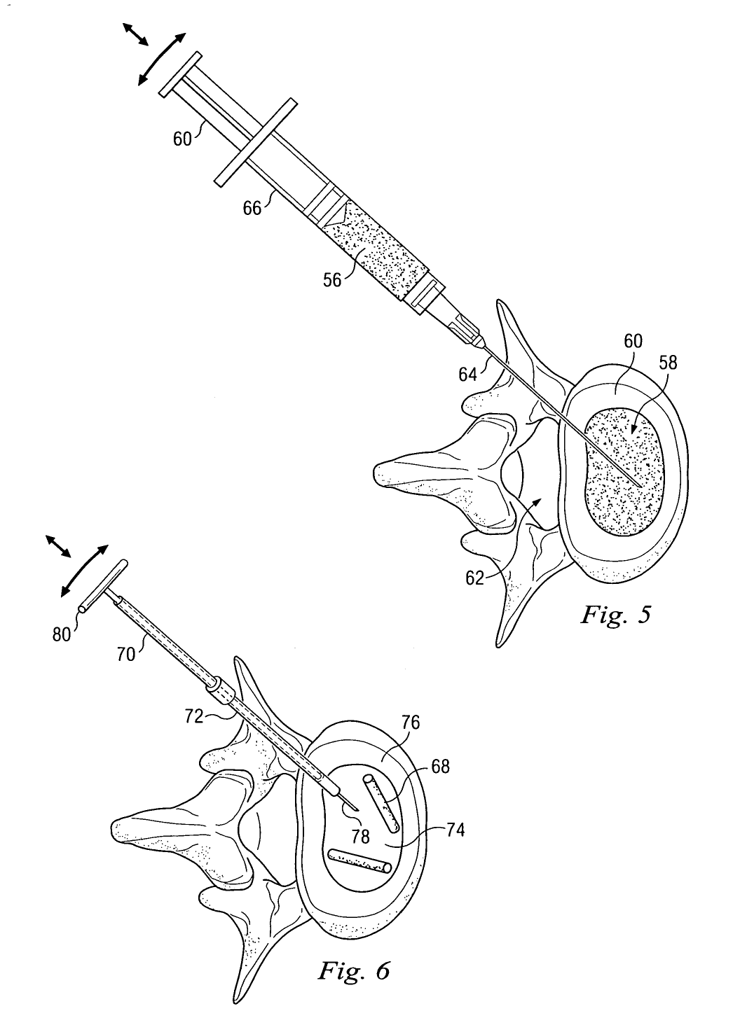

For example, referring now to FIG. 3, a biological treatment 30 may be injected into the joint capsule 32 of a facet joint 34 through a hypodermic needle 36 attached to a syringe 38. The syringe 38 is inserted into the joint capsule 32, and the syringe plunger 40 is depressed, thereby releasing the biological treatment into the joint capsule of the facet joint. As illustrated by the arrows in FIG. 3, the needle/syringe assembly may be moved around within the joint capsule, sweeping from side to side and back and forth, to ensure uniform distribution of the biological treatment within the facet joint. It is preferred, however, that the tip of the needle be maintained near the center of the joint capsule to ensure deposition of the material within the desired area, and to minimize potential leakage.

Referring now to FIG. 4, another method for injecting a biological treatment into a facet joint is illustrated. According to the embodiment illustrated in FIG. 4, a biological treatment 42 is provided in the form of microspheres, powders, particulates, pellets, granules, a plug, a solid, porous, woven or non-woven material. Biological treatment 42 may be compressed into a size suitable for delivery through a cannula 44 by pressure and/or heat and/or insertion through a small diameter tube. The delivery cannula 44 is attached to a dilator 46. The biological treatment 42 is inserted into a facet joint 48 by penetrating the capsule 50 of the facet joint with a guide needle 52. Dilator 46, preferably with delivery cannula 44 already attached, is inserted over guide needle 52. A plunger 54 may be used to push the biological treatment from the cannula into the facet joint. The form of the biological treatment may expand upon exiting the dilator, and may further expand as it hydrates or rehydrates in the facet joint.

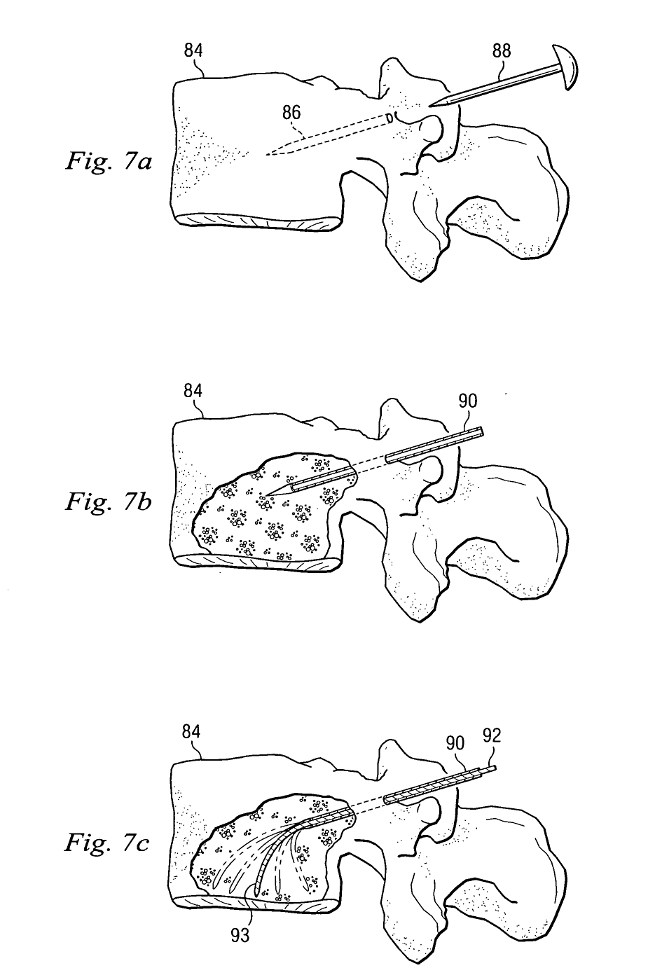

Referring now to FIG. 5, a method for injecting a biological treatment into a disc space is illustrated. According to the embodiment illustrated in FIG. 5, a biological treatment 56 may be injected into the nucleus pulposus 58 contained within a disc annulus 60 in an intervertebral disc space 62. Biological treatment 56 is injected through a hypodermic needle 64 attached to a syringe 66. The syringe 66 is inserted into the nucleus pulposus, and the syringe plunger 68 is depressed, thereby releasing the biological treatment into the disc space 62. As illustrated by the arrows in FIG. 5, the needle/syringe assembly may be moved around, sweeping from side to side and back and forth, to ensure uniform distribution of the biological treatment within the disc space. It is preferred, however, that the tip of the needle be maintained near the center of the disc space to ensure deposition of the material within the nucleus of the disc, and to minimize potential leakage.

Referring now to FIG. 6, another method for injecting a biological treatment into a disc space is illustrated. According to the embodiment illustrated in FIG. 6, a biological treatment 68 is provided in the form of granules, a plug, a solid, porous, woven or non-woven material. Biological treatment 68 may be compressed into a size suitable for delivery through a cannula 70 by pressure and/or heat and/or insertion through a small diameter tube. The delivery cannula 70 is attached to a dilator 72. The biological treatment 68 is inserted into the nucleus pulposus 74 by penetrating the annulus 76 of the disc with a guide needle 78. Dilator 72, preferably with delivery cannula 70 already attached, is inserted over guide needle 78. A plunger 80 may be used to push the biological treatment from the cannula into the nucleus pulposus. The form of the biological treatment may expand upon exiting the dilator, and may further expand as it hydrates or rehydrates.

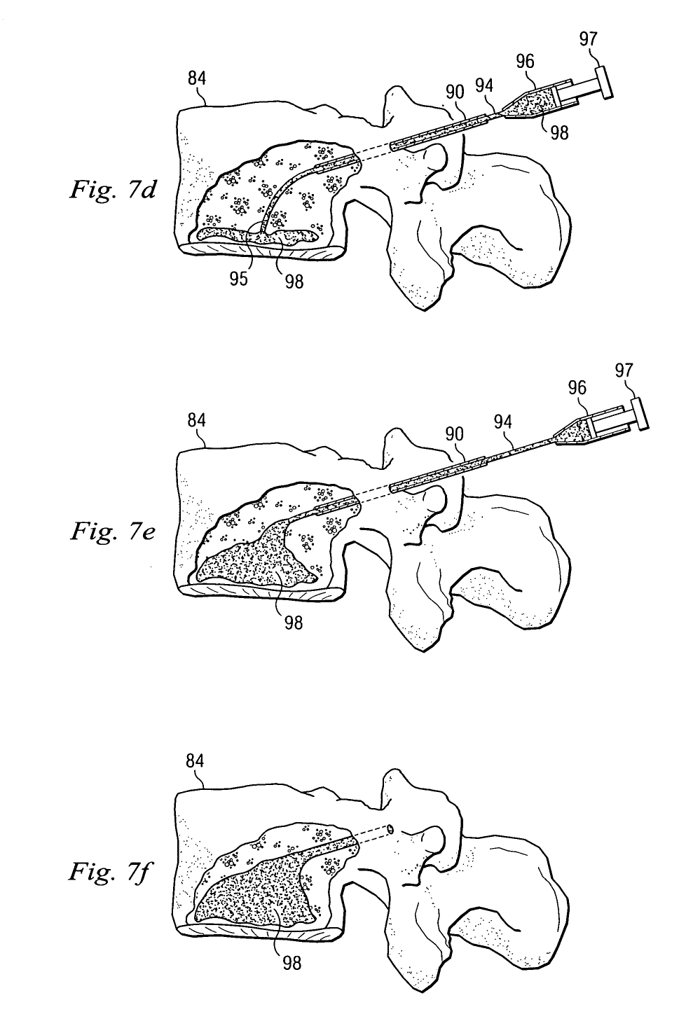

Referring now to FIGS. 7A-7F, a method of injecting a biological treatment into a vertebral body and/or an endplate is illustrated.

With reference now to FIG. 7A, a channel 86 can be created in vertebral body 84 through the pedicle using a suitable bone-penetrating implement such as a trocar needle 88. A sheath 90 (FIG. 7B) can be inserted into channel 86 through which various procedures can be implemented. FIG. 7C shows a subsequent step in which a flexible or otherwise steerable device 92, such as a needle or drill, is positioned through sheath 90 to access regions nearing the endplate of vertebral body 84. Although FIG. 7C illustrates positioning sheath 90 to access regions near the endplate of vertebral body 84, sheath 90 could also be positioned so as to access regions more central to the vertebral body itself, as opposed to the endplate.

Referring still to FIG. 7C, several directional passes of the steerable device 92 may be used in order to create access to a broader volume of bone. The tip 93 of steerable device 92 can be designed so as to be steerable, for instance by rotation of steerable device 92. As illustrated in FIG. 7D, after accessing near the endplate, (or to the vertebral body itself in other embodiments), the steerable device 92 can be withdrawn, and a delivery device 94 can be inserted through sheath 90. Delivery device 94 can have delivery tip 95, which is curved or otherwise steerable. Delivery device 94 can also include a reservoir 96 and a plunger 97, allowing for the delivery of a biological treatment 98 out of delivery tip 93. FIG. 7E shows an intermediate stage of the delivery process in which additional amounts of the biological treatment 98 are delivered as the sheath 90 and the delivery device 94 are withdrawn from the access channel 86. In this manner, the access channel 86 can be backfilled with the biological treatment 98 as the implements are withdrawn. Finally, shown in FIG. 7F is the biological treatment 98 occupying a volume overlying an endplate of the vertebral body 84, and also backfilled into the access channel 86.

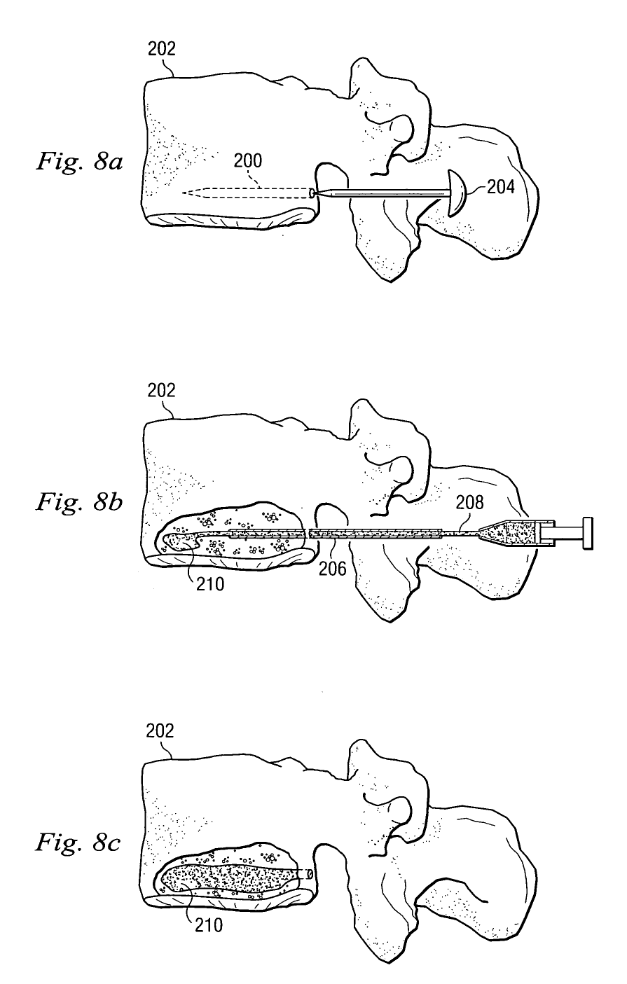

Referring now to FIGS. 8A-8C, another method of injecting a biological treatment into a vertebral body and/or an endplate is illustrated.

With reference now to FIG. 8A, an access channel 200 is created in vertebral body 202 just above the endplate using a bone-penetrating implement 204, for example, a needle. After this access, a sheath 206 is provided into channel 200. A delivery device 208 is then inserted through the lumen of sheath 206 and is used to deliver a biological treatment 210 into the vertebral body in a volume overlying the endplate. If desired or needed, a steerable needle or drill can be used to create access to a broader volume of bone, generally as described in conjunction with FIGS. 7A through 7F above. As well, a backfilling procedure can be used to fill the access channel 200 as the delivery device 208 and sheath 206 are removed. As shown in FIG. 8C, ultimately, a volume of the biological treatment 210 is delivered into the vertebral body overlying the endplate.

In other embodiments, a biological treatment may be introduced into an area of a vertebral column, such as a motion segment, through a needle/trocar assembly, as described in the above-referenced U.S. Patent Application Publication Nos. 2005/0031666. In still other embodiments, a biological treatment may be introduced into an area of a vertebral column by extrusion through a dilated annular opening, infusion through a catheter, insertion through an opening created by trauma or surgical incision, or by other means of invasive or minimally invasive deposition of materials into the area receiving the biological treatment.

According to certain embodiments described herein, when treating a vertebral column with a biological treatment, the load to be imposed on the treated area and/or on surrounding areas is also considered. For example, it can be noted whether the load imposed on a motion segment being treated would adversely affect the success of a biological treatment applied to the motion segment in achieving the desired repair, regeneration, reduction or prevention. By reducing the load imposed on the treated motion segment, the biological treatment is provided an opportunity to perform its function in an area that is less stressed, and therefore more receptive to the intended function of the biological treatment.

Thus, to achieve an improved clinical outcome and a stable result, biological treatments are applied in one or more of the anterior region, anterior column region, posterior region, and spinous process region of a vertebral column, while load-bearing devices and systems for treatment of one or more of the anterior region, anterior column region, posterior region, and spinous process region are also applied to provide a mechanical unloading of the region receiving the biological treatment.

Biological Treatment of Facet Joint and/or Disc Space Combined with Posterior Systems

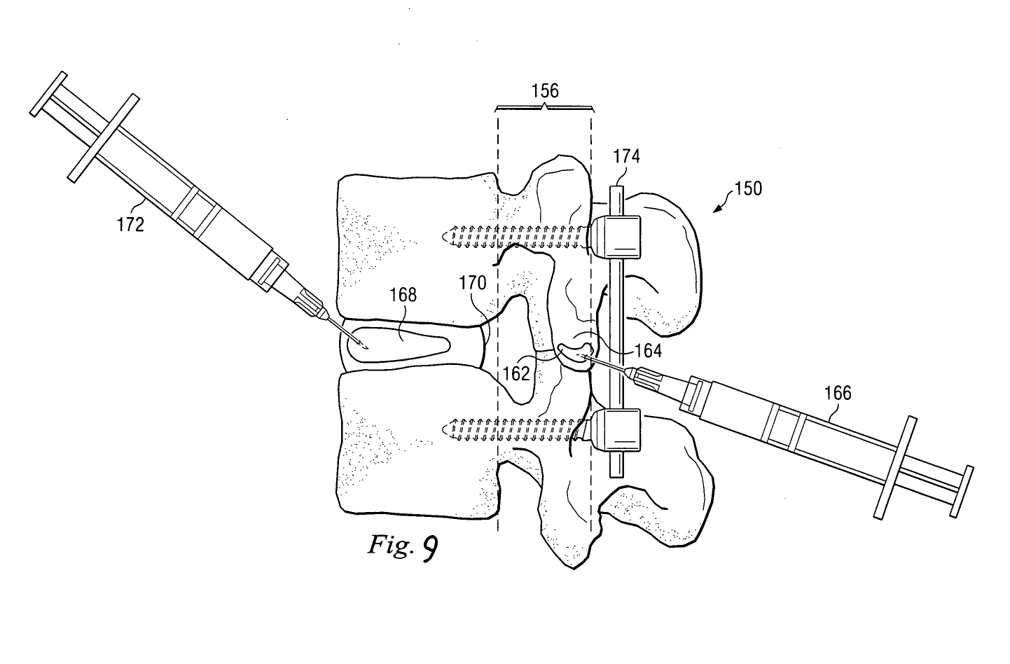

Referring now to FIG. 9, a combined treatment of a vertebral motion segment 150 with a biological treatment and a load-bearing device for treatment of the posterior region 156 of the spine is illustrated.

A biological treatment 162 has been applied to facet joint 164 by injection with an appropriately sized hypodermic needle 166. Selection of an appropriately sized hypodermic needle for injection into the facet joints of a spine is within the purview of one of ordinary skill in the art. Suitable methods for injecting the biological treatment 162 into the facet joint 164 are described above with respect to FIGS. 3 and 4. Other methods as described herein and as are known to those of ordinary skill in the art may also be used.

In the embodiment illustrated in FIG. 9, a biological treatment 168 has also been applied to disc space 170, which could include treatment of either or both of the nucleus and the annulus of the disc, with an appropriately sized hypodermic needle 172. Selection of an appropriately sized hypodermic needle for injection into the disc space is within the purview of one of ordinary skill in the art. Suitable methods for injecting the biological treatment 168 into the disc space 170 are described above with respect to FIGS. 5 and 6. Other methods as described herein and as are known to those of ordinary skill in the art may also be used.

Although two biological treatments 162 and 168 are illustrated, the present disclosure contemplates and includes application of just one biological treatment, or of two or more biological treatments. Moreover, biological treatments can be applied in other areas of the spine, for example, biological treatments can be applied to the anterior longitudinal ligament, the endplates, and the vertebral bodies.

According to the embodiment illustrated in FIG. 9, the treatment of facet joint 164 and the disc space 170 with biological treatments 162 and 168 is combined with a posterior device designed for treatment of the posterior region 156 of the vertebral motion segment 150. The posterior device is represented in FIG. 9 by posterior device 174, however the appearance of posterior device 174 is illustrative only, and it is understood that a wide variety of posterior devices could be used with the present embodiments.

According to some embodiments, a posterior device 174 may extend along the posterior or posterolateral side of the vertebral column and may span one or more vertebral motion segments.

In other embodiments, a posterior device 174 may be a rigid fixation system such as a hook, rod, or screw system, which are offered by or developed by Medtronic, Inc. of Minneapolis, Minn. under brands such as CD HORIZON, CD HORIZON SEXTANT, CD HORIZON M8, CD HORIZON LEGACY, CD HORIZON ANTARES, COLORADO 2, EQUATION, VERTEX, TSRH, and TSRH-3D.

In yet other embodiments, a posterior device 174 may be a semi-rigid or flexible system offered by or developed by Medtronic, Inc. under brand names such as FLEXTANT or AGILE, or offered by or developed by Zimmer, Inc. of Warsaw, Ind. such as the Dynesys® Dynamic Stabilization System. These types of flexible systems may be disclosed, for example, in U.S. Pat. Pub. Nos. 2005/0171540 and 2005/0131405. These particular systems may replace or supplement natural facet joints and may attach to the posterior features of adjacent vertebrae using bone screws.

Still other embodiments of a posterior device 174 include Archus Othopedics, Inc.'s TOTAL FACET ARTHROPLASTY SYSTEM (TFAS™) or similar devices performing facet functions. Still other embodiments of a posterior device 174 include facet repair devices such as described in U.S. Pat. No. 6,949,123, the entire disclosure of which is incorporated herein by reference.

According to still other embodiments, a posterior device 174 may be a dampener system, such as those described in U.S. Pat. Nos. 5,375,823; 5,540,688; 5,480,401 or U.S. Pat. App. Pub. Nos. 2003/0055427 and 2004/0116927, each of which is incorporated by reference herein.

In still another embodiment, posterior device 174 may include annulus repair or replacement devices for the posterior portion of the annulus. Additionally, posterior device 174 may also be a rod and screw system that uses flexible PEEK rods.

In still other embodiments, posterior device 174 may be made of flexible materials, such as woven or braided textile based devices that connect with two or more vertebrae. These flexible materials may be formed of natural graft material or synthetic alternatives. Posterior device 174 may also be formed of inelastic material, such as braided tethers or woven fabric of polyester or polyethylene, or of elastic material, such as rubber banding or plates, sheets, rods, or tubing made of silicone or polyurethane.

Posterior device 174 may be formed from biocompatible materials such as metals, polymers, ceramics, and tissue, and combinations thereof. For example, posterior device 174 may be formed from rigid materials such as titanium, titanium alloys, nickel titanium, tantalum, stainless steel, and combinations thereof. Alternatively, posterior device 174 may be formed of less rigid or more flexible materials such as polyaryletherketone (PAEK)-based materials, which includes polyetheretherketone (PEEK), polyetherketoneketone (PEKK), PEEK-carbon composite, etc., polyetherimide, polyimide, polysulfone, polyethylene, polyester, polylactide, copolymers of poly L-lactide and poly D-lactide, polyorthoester, tyronsine polycarbonate, polypolyurethane, silicone, etc. In some embodiments, the posterior device may be bioresorbable or partially resorbable.

Any of the foregoing posterior devices may be combined with any biological treatment. For example, in certain embodiments, a facet joint in a vertebral column receives a biological treatment. In one such embodiment, a biological treatment comprising injectable collagen containing stem cells and BMP-6 is applied to a facet joint in a vertebral column. A posterior device comprising a flexible system such as an AGILE brand system (Medtronic, Inc.) is applied to the posterior column region adjacent to the facet joint receiving the biological treatment to provide a mechanical unloading to the treated facet joint.

In other embodiments, a biological treatment comprising an injectable polyvinyl alcohol hydrogel containing chondrocytes and TGF-beta 2 is applied to a facet joint. A rod and screw system that uses flexible PEEK rods is applied to the posterior column region adjacent to the facet joint receiving the biological treatment to provide a mechanical unloading to the treated facet joint.

In still other embodiments, a biological treatment comprising injectable polyethylene glycol gel containing fibroblasts and TGF-beta is applied to a facet joint. A posterior device comprising a flexible system such as an AGILE brand system (Medtronic, Inc.) is applied to the posterior column region adjacent to the facet joint receiving the biological treatment to provide a mechanical unloading to the treated facet joint.

In yet other embodiments, a biological treatment comprising an injectable porcine-based collagen containing anti-TNF alpha and ILGF is applied to a facet joint. A posterior device comprising a flexible system such as a Dynesys® Dynamic Stabilization System (Zimmer, Inc.) is applied to the posterior column region adjacent to the facet joint receiving the biological treatment to provide a mechanical unloading to the treated facet joint.

According to still other embodiments, an intervertebral disc space receives a biological treatment. In one such embodiment, a biological treatment comprising injectable allogenic collagen containing stem cells, BMP-2 and BMP-6 is applied to an intervertebral disc. A posterior device comprising a flexible system such as an AGILE brand system (Medtronic, Inc.) is applied to the posterior column region adjacent to the disc receiving the biological treatment to provide a mechanical unloading to the treated disc space.

In other embodiments, a biological treatment comprising an injectable polyvinyl alcohol hydrogel containing stem cells and BMP-7 (OP-1) is applied to a disc space. A rod and screw system that uses flexible PEEK rods is applied to the posterior column region adjacent to the disc space receiving the biological treatment to provide a mechanical unloading to the treated disc space.

In still other embodiments, a biological treatment comprising injectable polyethylene glycol gel containing chondrocytes and TGF-beta 3 is applied to a disc space. A posterior device comprising a flexible system such as an AGILE brand system (Medtronic, Inc.) is applied to the posterior column region adjacent to the disc space receiving the biological treatment to provide a mechanical unloading to the treated disc space.

In yet other embodiments, a biological treatment comprising an injectable porcine-based collagen containing chondrocytes, TGF-beta 1 and PDGF is applied to a disc space. A posterior device comprising a flexible system such as a Dynesys® Dynamic Stabilization System (Zimmer, Inc.) is applied to the posterior column region adjacent to the disc space receiving the biological treatment to provide a mechanical unloading to the treated disc space.

Posterior device 174 may be connected to two or more vertebral bodies or vertebral endplates through the use of any connection mechanism such as bone screws, staples, sutures, or adhesives. The posterior device may be loaded in compression or tension depending upon the patient's indication or the performance of other implanted systems or treatments. For example, a flexible posterior device attached to adjacent vertebrae with bone screws may be installed in compression to reduce the load on the disc space 170 or facet joint 164 where a biological treatment was applied.

According to one embodiment, a procedure for performing the methods described herein includes surgically accessing at least a portion of a patient's spine, and implanting a load-bearing device in the patient's spine. In one aspect, the load-bearing device is implanted so as to mechanically unload all or a portion of the facet joint and/or the disc space, which receives a biological treatment. In another aspect, the load-bearing device is implanted into an area of the spine that is intact, for example, a motion segment where the anatomy has not been surgically disrupted. In yet another aspect, the anatomy of the area of the spine in which the load-bearing device is being implanted has been surgically disrupted, for example, a resection of the facet or the spinous process, or even a discectomy, has been performed.

Whether the spinal anatomy is intact or has been disrupted, the load-bearing device is implanted in to the spine in a position so as to be at least partially load-bearing with respect to the area that is to receive a biological treatment. The device thus mechanically unloads all or a portion of the area to receive the biological treatment. For example, a rigid fixation system may be placed on the posterior portion of the spine to transfer load away from the disc space and/or the facets. In one aspect, the facet joints and/or the adjacent vertebral bodies defining the disc space are mechanically moved by placement of the mechanical unloading device to align the facet joint and/or increase the distance between the adjacent vertebral bodies. After application of the mechanical unloading device, a biological treatment is performed on at least one facet and/or the disc space. In another aspect, the above-described steps may be reversed such that the biological treatment of the facet joint and/or the disc space occurs first, and the mechanical unloading occurs later.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

For example, each of the following patent applications are incorporated herein by reference, as each describes spinal devices that can be applied to the anterior, anterior column, posterior, or spinous process regions of the vertebral column, and that can be used to unload an area treated with a biological treatment as described herein.

| | Attorney | | |
|---|---|---|---|
| Title | Docket No. | Filing Date | Inventor(s) |
| Materials, Devices, and | P22656.00 | Jan. 13, 2006 | Hai H. Trieu |
| Methods for Treating | 31132.378 | | |
| Multiple Spinal Regions | | | |
| Including The Interbody | | | |
| Region | | | |
| Materials, Devices, and | P22578.00 | Jan. 13, 2006 | Hai H. Trieu |
| Methods for Treating | 31132.376 | | |
| Multiple Spinal Regions | | | |
| Including The Posterior | | | |
| and Spinous Process | | | |
| Regions | | | |
| Materials, Devices, and | P22615.00 | Jan. 13, 2006 | Hai H. Trieu |
| Methods for Treating | 31132.377 | | |
| Multiple Spinal Regions | | | |
| Including The Anterior | | | |
| Region | | | |
| Materials, Devices, and | P22681.00 | Jan. 13, 2006 | Hai H. Trieu |
| Methods for Treating | 31132.379 | | |
| Multiple Spinal Regions | | | |
| Including Vertebral Body | | | |
| and Endplate Regions | | | |
| Use Of A Posterior | P22397.00 | Jan. 13, 2006 | Aure Bruneau |
| Dynamic Stabilization | 31132.420 | | et al. |
| System With An | | | |
| Interdiscal Device | | | |

In addition, each of the following applications describes suitable biological treatments that can be applied to an area of the vertebral column, and spinal devices that can be applied to the anterior, anterior column, posterior, or spinous process regions of the vertebral column to unload the treated area. Each of the following applications was filed concurrently with the present application, assigned to the same assignee, and each is hereby incorporated by reference.

| | Attorney | | |
|---|---|---|---|
| Title | Docket No. | Filing Date | Inventor(s) |
| Treatment of the | P23559.00 | concurrent with | Hai H. Trieu |
| Vertebral Column | 31132.477 | the present | |
| | | application | |
| Treatment of the | P23556.00 | concurrent with | Hai H. Trieu |
| Vertebral Column | 31132.474 | the present | |
| | | application | |
| Treatment of the | P23558.00 | concurrent with | Hai H. Trieu |
| Vertebral Column | 31132.476 | the present | |
| | | application | |
| Biological Fusion | P23568.00 | concurrent with | Hai H. Trieu |
| in the Vertebral | 31132.478 | the present | Mike Sherman |
| Column | | application | |
| Treatment of the | P23598.00 | concurrent with | Hai H. Trieu |
| Vertebral Column | 31132.479 | the present | |
| | | application | |

It is understood that all spatial references, such as “horizontal,” “vertical,” “top,” “inner,” “outer,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A method of treating a vertebral column comprising:
introducing a biological treatment into at least one area of a vertebral column selected from a facet joint, a disc space, a vertebral body and an endplate; and
at least partially mechanically unloading the treated area by applying a load-bearing device to at least a posterior region of the vertebral column.

2. The method of claim 1 wherein the biological treatment comprises a biologically active component.

3. The method of claim 2 wherein the biological treatment further comprises a biological additive.

4. The method of claim 3 wherein the biological additive comprises at least one of a biomaterial carrier, a therapeutic agent, a liquid and a lubricant.

5. The method of claim 3 wherein the biological additive is selected from autogenic collagen, allogenic collagen, xenogenic collagen, human recombinant collagen, gelatin, hyaluronic acid, fibrin, albumin, keratin, silk, elastin, glycosaminoglycans (GAGs), polyethylene glycol (PEG), polyethylene oxide (PEO), polyvinyl alcohol (PVA) hydrogel, polyvinyl pyrrolidone (PVP), co-polymers of PVA and PVP, polysaccharides, platelet gel, peptides, carboxymethyl cellulose, modified starches and celluloses.

6. The method of claim 3 wherein the biological additive is selected from nutrients, analgesics, antibiotics, anti-inflammatories, steroids, antiviricides, vitamins, amino acids and peptides.

7. The method of claim 6 wherein the biological additive comprises at least one of:
an analgesic selected from codeine, prodrugs, morphine, hydromorphone, propoxyphene, hydrocodone, oxycodone, meperidine, methadone, and fentanyl; and
an antibiotic selected from erythromycin, bacitracin, neomycin, penicillin, polymyxin B, tetracyclines, viomycin, chloromycetin, streptomycins, cefazolin, ampicillin, azactam, tobramycin, clindamycin and gentamycin.

8. The method of claim 3 wherein the biological additive is selected from water, saline, radio-contrast media, hyaluronic acid, a salt of hyaluronic acid, sodium hyaluronate, glucosaminoglycan, dermatan sulfate, heparin sulfate, chondroitin sulfate, keratin sulfate, synovial fluid, a component of synovial fluid, vitronectin and rooster comb hyaluronate.

9. The method of claim 1 wherein the biological treatment comprises a biologically active component selected from anti-cytokines; cytokines; anti-interleukin-1 components (anti-IL-1); anti-TNF alpha; growth factors; LIM mineralization proteins; stem cell material, autogenic chondrocytes; allogenic chondrocytes; autogenic chondrocytes with one of a retroviral viral vector or a plasmid viral vector; allogenic chondrocytes with one of a retroviral viral vector or a plasmid viral vector; and fibroblasts.

10. The method of claim 1 wherein the biological treatment comprises a biologically active component selected from transforming growth factors, bone morphogenetic proteins, fibroblast growth factors, platelet derived growth factor (PDGF), insulin-like growth factor (ILGF); human endothelial cell growth factor (ECGF); epidermal growth factor (EGF); nerve growth factor (NGF); and vascular endothelial growth factor (VEGF).

11. The method of claim 10 wherein the biologically active component comprises at least one of a transforming growth factor selected from TGF-beta 1, TGF-beta 2, and TGF-beta 3, and a bone morphogenetic protein selected from BMP-2, BMP-3, BMP-4, BMP-6, BMP-7, and BMP-9.

12. The method of claim 1 wherein the biological treatment comprises stem cell material selected from dedifferentiated stem cells, undifferentiated stem cells, mesenchymal stem cells, marrow-extracted stem cell material and adipose-derived stem cell material.

13. The method of claim 1 wherein the biological treatment comprises a biologically active component selected from cartilage derived morphogenetic protein (CDMP);
cartilage inducing factor (CIP); proteoglycans; hormones; and matrix metalloproteinases (MMP) inhibitors.

14. The method of claim 1 wherein the biological treatment comprises a biologically active component selected from allogenic disc annulus material, xenogenic disc annulus material, biologic tissues, activated tissue grafts, engineered cells comprising a nucleic acid for encoding a protein or variant thereof, and a recombinant human bone morphogenetic protein.

15. The method of claim 1 wherein the load-bearing device is formed from a material selected from polyaryletherketone (PAEK), polyetheretherketone (PEEK), polyetherketoneketone (PEKK), PEEK-carbon composite, polyetherimide, polyimide, polysulfone, polyethylene, polyester, polylactide, copolymers of poly L-lactide and poly D-lactide, polyorthoester, tyrosine polycarbonate, polyurethane, silicone and polyolefin rubber, and combinations thereof.

16. The method of claim 1 wherein the load-bearing device is bioresorbable or partially resorbable.

17. The method of claim 1 wherein the load-bearing device comprises at least one of a fixation system, a dampener system, an annulus repair system, and an annulus replacement system.

18. The method of claim 17 wherein the load-bearing device comprises a fixation system comprising at least one of hooks, rods and screws.

19. The method of claim 1 wherein the load-bearing device comprises at least one of plates, sheets and rods.

20. The method of claim 1 wherein the load-bearing device is formed from a biocompatible material selected from metals, polymers, ceramics, and tissue, and combinations thereof.

21. The method of claim 1 wherein:
the area of the vertebral column receiving the biological treatment comprises a facet joint.

22. The method of claim 1 wherein:
the area of the vertebral column receiving the biological treatment comprises a facet joint;
the biological treatment comprises collagen, stem cells and BMP-6; and
the load-bearing device is applied to the posterior region adjacent to the facet joint receiving the biological treatment.

23. The method of claim 1 wherein:
the area of the vertebral column receiving the biological treatment comprises a facet joint;
the biological treatment comprises a polyvinyl alcohol hydrogel containing chondrocytes and TGF-beta 2; and
the load-bearing device comprises a fixation system comprising at least one rod and at least one screw applied to the posterior region adjacent to the facet joint receiving the biological treatment.

24. The method of claim 1 wherein:
the area of the vertebral column receiving the biological treatment comprises a facet joint;
the biological treatment comprises a polyethylene glycol gel containing fibroblasts and TGF-beta; and
the load-bearing device is applied to the posterior region adjacent to the facet joint receiving the biological treatment.

25. The method of claim 1 wherein:
the area of the vertebral column receiving the biological treatment comprises a facet joint;
the biological treatment comprises a porcine-based collagen containing anti-TNF alpha and ILGF; and
the load-bearing device is applied to the posterior region adjacent to the facet joint receiving the biological treatment.

26. The method of claim 1 wherein the biological treatment is non load-bearing.

27. A method for treating a motion segment of a spinal column comprising:
accessing a portion of a patient's spinal column;
implanting a load-bearing device to at least partially mechanically unload an intact motion segment in the patient's spinal column, wherein the intact motion segment comprises a facet joint; and
injecting a biological treatment into the facet joint.

28. The method of claim 27 wherein the load-bearing device is implanted at a location in the spine that is spaced from the motion segment receiving the biological treatment.

29. The method of claim 27 wherein the load-bearing device is implanted at a location in the spine that is adjacent to the motion segment receiving the biological treatment.

30. The method of claim 27 wherein the biological treatment is non-load-bearing.
